(19) United States
(12) Patent Application Publication
(10) Pub. No.: US 2011/0165150 A1
(43) Pub. Date: Jul. 7, 2011

(54) Isolated organ perfusion combination therapy of cancer

(75) Inventors: Simon Goodman, Griesheim (DE); Matthias Grell, Darmstadt (DE); Timo L.M. Ten Hagen, Rotterdam (NL); Alexander M.M. Eggermont, Rotterdam (NL)

(73) Assignee: Merck Patent GmbH, Danmstadt (DE)

(21) Appl. No.: 12/161,465

(22) PCT Filed: Jan. 18, 2006
(86) PCT No.: PCT/EP2007/000408

§ 371 Date: Jul. 18, 2008

(30) Foreign Application Priority Data
Jan. 18, 2006 (EP) 06001049.3

(51) Int. Cl. A61K 39/395; A61K 38/00; A61K 38/08; A61K 33/24; A61P 35/00

(52) U.S. Cl. 424/133.1; 514/19.3; 424/649

(57) ABSTRACT

The invention relates to a combination therapy for the treatment of tumors and tumor metastases comprising administration of integrin ligands, preferably integrin antagonists, together with co-therapeutic agents or therapy forms that have synergistic efficacy when administered together with said ligands, such as chemotherapeutic agents and/or radiation therapy, in isolated organ perfusion. The therapy results in a synergistic potential increase of the inhibition effect of each individual therapeutic on tumor cell proliferation, yielding more effective treatment than found by administering an individual component alone.

### TECHNICAL FIELD OF THE INVENTION

The invention relates to a combination therapy for the treatment of tumors and tumor metastases comprising administration of integrin ligands together with cancer-cotherapeutic agents or other cancer cotherapeutic therapy forms that have additive or synergistic efficacy when administered together with said integrin ligand, such as chemotherapeutic agents, immunotherapeutics, including antibodies, radioimmunoconjugates and immunocytokines and/or radiation therapy, via isolated organ perfusion. The therapy preferably results in a synergistic potential increase of the inhibition effect of each individual therapeutic on tumor cell and tumor endothelial cell proliferation, yielding more effective treatment than found by administering an individual component alone, and preferably also a more effective treatment than the combinations of prior art.

### BACKGROUND OF THE INVENTION

Vascular endothelial cells are known to contain at least three RGD-dependent integrins, including the vitronectin receptors αᵥβ₃ or αᵥβ₅ as well as the collagen types I and IV receptors α′ᵥβ₁ and α₂β₁, the laminin receptors α₆β₁ and α₃β₁, and the fibronectin receptor α₅β₁ (Davis et al., 1993, J. Cell. Biochem. 51, 206). The smooth muscle cell is known to contain at least six RGD-dependent integrins, including αᵥβ₃ and αᵥβ₅.

Inhibition of cell adhesion in vitro using monoclonal antibodies immunospecific for various integrin α or β subunits have implicated the vitronectin receptor αᵥβ₃ in cell adhesion processes of a variety of cell types including microvascular endothelial cells (Davis et al., 1993, J. Cell. Biol. 51, 206).

Integrins are a class of cellular receptors known to bind extracellular matrix proteins, and mediate cell-extracellular matrix and cell-cell interactions, referred generally to as cell adhesion events. The integrin receptors constitute a family of proteins with shared structural characteristics of non-covalenty associated heterodimeric glycoprotein complexes formed of α and β subunits. The vitronectin receptor, named for its original characteristic of preferential binding to vitronectin, is now known to refer to four different integrins, designated αᵥβ₁, αᵥβ₃, αᵥβ₅ and αᵥβ₈. αᵥβ₁ binds fibronectin and vitronectin. αᵥβ₃ binds a large variety of ligands, including fibrin, fibrinogen, laminin, thrombospondin, vitronectin and von Willebrand's factor. αᵥβ₅ binds vitronectin. It is clear that there are different integrins with different biological functions as well as different integrins and subunits having shared biological specificity and function. One important recognition site in a ligand for many integrins is the Arg-Gly-Asp (RGD) tripeptide sequence. RGD is found in all of the ligands identified above for the vitronectin receptor integrins. The molecular basis of RGD recognition by αᵥβ₃ has been identified (Xiong et al., 2001). This RGD recognition site can be mimicked by linear and cyclic(poly)peptides that contain the RGD sequence. Such RGD peptides are known to be inhibitors or antagonists, respectively, of integrin function. It is important to note, however, that depending upon the sequence and structure of the RGD peptide, the specificity of the inhibition can be altered to target specific integrins. Various RGD polypeptides of varying integrin specificity have been described, for example, by Cheresh, et al., 1989, Cell 58, 945, Aumailley et al., 1991, FEBS Letts. 291, 50, and in numerous patent applications and patents (e.g. U.S. Pat. Nos. 4,517,686, 4,578,079, 4,589,881, 4,614,517, 4,661,111, 4,792,525; EP 0770 622).

The generation of new blood vessels, or angiogenesis, plays a key role in the growth of malignant disease and this has generated much interest in developing agents that inhibit angiogenesis.

Nevertheless, although various combination therapies utilizing potential angiogenesis inhibitors are under investigation, in clinical trials and on the market, the outcome of these therapies are not sufficiently fruitful. Therefore, there still exists a need in the art to develop further combinations which can show increased efficacy and reduced side-effects.

It is known today that tumor vasculature is different from vasculature of healthy tissue. The vasculature is characteristic for the tumor and distinct from the stable, dormant vasculature of healthy tissue. It is often characterized by an increased expression and priming of specific cell adhesion molecules of the alpha-v-integrin series, especially αᵥβ₃ and αᵥβ₅. When activated these integrins enhance the cellular response to growth factors that drive angiogenesis, for example VEGFA and FGF2: VEGFA is originally termed vascular permeability factor, and it acts via the SRC kinase pathway to increase local vascular permeability. VEGRF2, when activated, increases the activity of αᵥβ₃ integrin.

Soft tissue sarcoma and malignant melanoma often metastasize to peripheral limbs and require surgical intervention.

Hepatocellular carcinoma (HCC) is a major problem in the developing world, and a growing problem in the developed world. Often the initiators are viruses like Hepatitis C virus. There is no effective treatment for HCC nor a non-surgical option for distributed soft tissue sarcoma or malignant melanoma. However, a current experimental clinical rescue therapy for distributed malignant melanoma, soft tissue sarcoma and/or breast carcinoma, and a local therapy of HCC involves the isolated perfusion of the organ or limb, e.g. the liver (IHP), with high concentrations of an alkylating cytotoxic, such as melphalan. The efficacy of this therapy however is low. It has a weak, rescue effect, only, and can be compared with a similar protocol used in isolated limb perfusion for soft tissue sarcoma and distributed malignant melanoma.

The metastatic process is a multistep event and represents the most dreadful aspect of cancer. At the moment of diagnosis, cancers are frequently far advanced in their natural history, and the presence of metastases is a common event. In fact, approximately 30% of patients have detectable metastases at the moment of clinical diagnosis and a further 30% of patients have occult metastases. Metastases can be disseminated and they can infest different organs at the same time, or localize to a specific organ. In the case of localized disease, surgery is the treatment of choice; however recurrence and prognosis depend on many criteria such as: resectability, patient's clinical situation, and number of metastases.

After resection, recurrence is common, suggesting that micrometastatic foci are present at the moment of diagnosis. Systemic chemotherapy is an ideal setting but only few patients are cured by it, and in the majority systemic chemotherapy fails. Many physiological barriers and pharmacokinetic parameters contribute to decrease its efficacy.

Systemic chemotherapy compared to regional chemotherapy has limited benefits. Regional chemotherapy consists in the isolation of an anatomical region and in treating this by using chemotherapy at high doses with absent or minimal systemic toxicity. Typical indications where regional chemotherapy has been used are limbs, lung, liver, pleura, pelvis and pancreas. The method is common to all organs and consists in different sequential steps. The first step is the surgical isolation of the organ; the second is to keep the organ perfused. In brief, the perfusion is maintained by a circuit that consists of out and inflow catheters, tubing, a roller pump, a reservoir, a heat exchanger and an oxygenator. Often, hyperthermia is applied to increase the sensitivity of tumor cells to antineoplastic agents, to kill more tumor cells and so lowering recurrence. Aside from limbs, for which isolated chemohyperthermia is now an accepted treatment, the organs actually treated with perfusion chemohyperthermia are: lung, pleura and liver. The lung is the most common site of metastatic involvement beside the lymph nodes for all cancer types. Lung metastases occur in 50% of patients with cancer diagnosis. Retroprospective studies have demonstrated that surgical removal, with an aggressive approach in selected patients, is the treatment of choice. However, technical and clinical limitations exist. Patients with unresectable lung metastases are candidates for isolated lung perfusion chemotherapy. This causes, as described above, a more efficient drug delivery to lung tissue. Animals' studies have demonstrated a superiority of perfusion technique compared to systemic chemotherapy. The Johnston and Ratto groups used cisplatinum and demonstrated a high concentration of the drug inside the diseased lung (M R. Johnston, R F. Minchen, and C A. Dawson. “Lung perfusion with chemotherapy in patients with unresectable metastatic sarcoma to the lung or diffuse bronchioalveolar carcinoma.”, J. Thorac. Cardiovasc. Surg. 1995, 110: 368-373; G B. Ratto, S. Toma, and D. Civalleri. “Isolated lung perfusion with platinum in the treatment of pulmonary metastases from soft tissue sarcomas.” J. Thorac. Cardiovasc. Surg. 1996, 112: 614-622). Other authors used or are using other antineoplastic agents such as melphalan, doxorubicin and TNFα.

Liver, lungs and lymph nodes are filtration organs and therefore inclined to metastasization. The poor chemosensitivity of metastases, peculiarly those of colorectal origin has forced many researchers to use methods for increasing the time and the concentration of drugs. The need for decreasing or limiting the side effects for this important and delicate organ led to the development of the technique of liver isolation for perfusion of antineoplastic agents. (K. R. Aigner, Isolated liver perfusion. In: Morris D L, McArdle C S, Onik G M, eds. Hepatic Metastases. Oxford: Butterworth Heinemann, 1996. 101-107). Since 1981, modifications and technical improvements have been continuously introduced. Liver metastases may be of different origin and their chemosensitivity may vary according to the histological type and their response in presence of heat.

A detailed summary of perfusion treatment can be found e.g. in the Eureka Bioscience Collection, Lands Bioscience, available as an online book from the NCBI databases.

Since 1993, 358 patients with hepatic metastases have been treated with IHP. The results of these clinical trials have been recently reviewed by Grover and Alexander. On 15 trials reported, the majority have been conducted with melphalan alone or in association with cisplatin or TNFα, 12 of them with metastases from colorectal cancer, 3 malignant melanomas and five with mixed histology. Some authors used mitomicyn-C for the treatment of colorectal metastases for the known synergistic effect with hyperthermia. With this procedure a partial response of 41% has been obtained, and a complete response of 6-9%. The median survival time has been 10 months. See e.g. A. Grover and H R. Alexander. “The past decade of experience with isolated hepatic perfusion.” The Oncologist, 2004. 9: 653-664; A L. Vahrmeijer, J K. van Dierendonck, and H J. Keizer. “Increased local cytostatic drug exposure by isolated hepatic perfusion: a phase I clinical and pharmacologic evaluation of treatment with high dose melphalan in patients with colorectal cancer confined to the liver.” Br. J. Cancer, 2000. 82: 1536-1546; P. Lindenér, M. Fjälling, and L. Hafström. “Isolated hepatic perfusion with extracorporeal oxygenation using hyperthermia, tumor necrosis factor alpha and melphalan.” European J. of Surgical Oncology, 1999. 25: 179-185; A. Marinelli, L M. de Brau, and H. Beerman. “Isolated liver perfusion with mitomycin-c in the treatment of colorectal cancer metastases to the liver.” Jpn. J. Clin. Oncol., 1996. 26: 341-350.

Even with such sophisticated technology of isolated organ perfusion at hand there still exists a growing need in the art in order to develop new therapeutic strategies for treating cancer, especially metastases, especially in limbs, lung, liver, pleura and pancreas, possibly kidney or pelvis and other organs. The object of the present invention therefore was to develop such a new strategy. It should be applicable to isolated organ perfusion, and it should further lower the dose and/or increase the efficiency of the cancer therapeutical agent to be applied.

### SUMMARY OF THE INVENTION

The present inventions describe for the first time a novel pharmaceutical treatment which is based on the new concept in tumor therapy to administer to an individual in a therapeutically effective amount an integrin ligand together with the application of a cancer cotherapeutic agent in isolated organ perfusion, wherein the said application may be prior, concurrent or subsequent to the integrin ligand administration. The subsequent application is preferred. Equally preferred is the concurrent application.

In one embodiment the present invention relates to a composition comprising as the cotherapeutic agent therapeutically active compounds, preferably selected from the group consisting of cytotoxic agents, chemotherapeutic agents and immunotoxic agents, and as the case may be other pharmacologically active compounds which may enhance the efficacy of said agents or reduce the side effects of said agents in isolated organ perfusion.

Thus, in one embodiment the present invention relates to pharmaceutical compositions for isolated organ perfusion comprising as preferred integrin ligand any of the αᵥβ₃, αᵥβ₅, αᵥβ₆ or αᵥβ₈ integrin receptor ligands, preferably an RGD-containing linear or cyclic peptide, preferably RGD-containing integrin inhibitors, most preferably with the cyclic peptide cyclo-(Arg-Gly-Asp-DPhe-NMe-Val), and/or the pharmaceutically acceptable derivatives, solvates and salts thereof, optionally together with one or more cancer cotherapeutic agents, preferably a cancer cotherapeutic agent, for example selected from the group consisting of chemotherapeutic, immunotoxic and cytotoxic compounds.

According to this invention therapeutically active agents may preferably also be provided by means of a pharmaceutical kit comprising a package comprising one or more of the said integrin ligands, and optionally one or more cytotoxic and/or chemotherapeutic and/or immunotoxic agents in single packages or in separate containers. The therapy with these combinations may include optionally treatment with radiation with or without a further cotherapeutic agent as defined above.

The invention relates furthermore to a combination therapy comprising the administration of only one molecule, having integrin ligand activity together with radiotherapy prior to, together with, or after the application of the integrin ligand.

It is therefore a further preferred embodiment of the present invention if the integrin ligand is administered in combination with radiotherapy, only. In this context, according to the present invention, radiation, or, radiotherapy preferably has to be understood as a cancer cotherapeutic agent.

It should be understood that the administration of any combination of the present invention can preferably be accompanied by radiation therapy, wherein radiation treatment can be done substantially concurrently, before or after the administration. The administration of the different agents of the combination therapy according to the invention can also be achieved substantially concurrently or sequentially. Preferably, the administration of the specific integrin ligand takes place prior or substantially concurrently, preferably prior, to the administration of the one or more cancer cotherapeutic agents. More preferably, the administration of the specific integrin ligand takes place prior or substantially concurrently, preferably prior, to the administration of the administration of the radiotherapy, even more preferably in a timed administration as described herein. This timed administration is preferably also referred to as “timed and combined administration”.

It is known that tumors elicit alternative routes for their development and growth. If one route is blocked they often have the capability to switch to another route by expressing and using other receptors and signaling pathways. Therefore, the pharmaceutical combinations of the present invention may block several of such possible development strategies of the tumor and provide consequently various therapeutic benefits. The combinations according to the present invention are useful in treating and preventing tumors, tumor-like and neoplasia disorders and tumor metastases, which develop and grow by activation of their relevant hormone receptors which are present on the surface of the tumor cells.

Preferably, the different combined agents of the present invention are administered in combination at a low dose, that is, at a dose lower than has been conventionally used in clinical situations. A benefit of lowering the dose of the compounds, compositions, agents and therapies of the present invention administered to an individual includes a decrease in the incidence of adverse effects associated with higher dosages. For example, by the lowering the dosage of an agent described above and below, a reduction in the frequency and the severity of nausea and vomiting will result when compared to that observed at higher dosages. By lowering the incidence of adverse effects, an improvement in the quality of life of a cancer patient is expected. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects. Alternatively, the methods and combination of the present invention can also maximize the therapeutic effect at higher doses.

Tumors, preferably showing an increased expression, a priming and/or activation of specific cell adhesion molecules of the alpha-v-integrin series, especially αᵥβ₃ and αᵥβ₅ in their vasculature may be successfully treated by the combinations and therapeutic regimen according to the invention. The combinations within the pharmaceutical treatment according to the invention show an astonishing synergetic effect. In administering the combination of drugs real tumor shrinking and disintegration could be observed during clinical studies while no significant adverse drug reactions are detectable.

Preferred embodiments of the present invention relate to:

A combination therapy unit for the timed and combined use as a combination therapy for the treatment of cancer via isolated organ perfusion, the unit comprising

a) a composition containing at least one specific integrin ligand, the unit further comprising

b) at least one further cancer-cotherapeutic agent different from the at least one specific integrin ligand of a).

A said unit wherein the at least one integrin ligand is selected from the group consisting of αᵥ integrin inhibitors, preferably αᵥβ₃ inhibitors, mostly preferred cyclo-(Arg-Gly-Asp-DPhe-NMeVal).

A said unit wherein the at least one cancer-cotherapeutic agent is selected from the group consisting of chemotherapeutical agents, cytotoxic agents, immunotoxic agents and radiotherapy.

A said unit wherein the isolated organ is selected from the group consisting of limbs, lung, liver, pleura, pancreas, kidney or pelvis.

A said unit wherein the isolated organ is liver and the cancer to be treated is hepatocellular carcinoma.

A said unit wherein the at least one further cancer-cotherapeutic agent different from the at least one specific integrin ligand of a) is radiotherapy.

A method for the treatment of cancer characterized in treating via isolated organ perfusion a subject in need thereof with a therapeutically effective amount of at least one integrin ligand and at least one cancer-cotherapeutic agent.

A said method wherein the at least one integrin ligand is selected from the group consisting of αᵥ integrin inhibitors, preferably αᵥβ₃ inhibitors, mostly preferred cyclo-(Arg-Gly-Asp-DPhe-NMeVal).

A said method wherein the at least one cancer-cotherapeutic agent is selected from the group consisting of chemotherapeutical agents, cytotoxic agents, immunotoxic agents and radiotherapy.

A said method wherein the isolated organ is selected from the group consisting of limbs, lung, liver, pleura, pancreas, kidney or pelvis.

A said method wherein the isolated organ is liver and the cancer to be treated is hepatocellular carcinoma.

Set for use in isolated organ perfusion for the treatment of cancer comprising independent dosage forms of:

a) a therapeutically effective amount of at least one integrin ligand preferably being selected from the group consisting of αᵥ integrin inhibitors, preferably αᵥβ₃ inhibitors, mostly preferred cyclo-(Arg-Gly-Asp-DPhe-NMeVal), and

—provided that the at least one further cancer-cotherapeutic agent of b) is not radiotherapy—

b) a therapeutically effective amount of at least one further cancer-cotherapeutic agent different from the integrin ligand of a), selected from the group consisting of chemotherapeutical agents, cytotoxic agents, immunotoxic agents.

A said set wherein the organ is liver and the cancer is hepatocellular carcinoma.

A said set, the at least one further cancer-cotherapeutic agent being radiotherapy.

Said set is further characterized in that it will be advantageous to give detailed instructions to and how to use radiotherapy in connection with the integrin ligand in form of a specific packaging, specific package inserts and similar.

Therefore, a further preferred embodiment of the present invention is a medicament consisting of an integrin ligand as one active ingredient, designed to be applied prior, concurrently or after radiotherapy, and contained in a container or similar, the container giving in form of writing detailed instructions and/or technical information on how to use said medicament in combination with radiotherapy.

The use of at least one integrin ligand and at least one cancer-cotherapeutic agent for the preparation of a medicament for the treatment of cancer via isolated organ perfusion, the at least one integrin ligand preferably being selected from the group consisting of αᵥ integrin inhibitors, preferably αᵥβ₃ inhibitors, mostly preferred cyclo-(Arg-Gly-Asp-DPhe-NMeVal) and the cancer-cotherapeutic agent being selected from the group consisting of chemotherapeutical agents, cytotoxic agents and/or immunotoxic agents A said use wherein the organ is liver and the cancer is hepatocellular carcinoma.

A preferred embodiment of the present invention relates to a corresponding pharmaceutical composition for use in isolated organ perfusion, wherein the said integrin ligand is an αᵥβ₃, αᵥβ₅, αᵥβ₆ or αᵥβ₈ integrin inhibitor; a corresponding pharmaceutical composition, wherein said integrin inhibitor is an RGD-containing linear or cyclic peptide; and, as a specific and very preferred embodiment, a said pharmaceutical composition, wherein said integrin ligand is cyclo(Arg-Gly-Asp-DPhe-NMeVal), comprising optionally in separate containers or packages, a chemotherapeutic agent selected from any of the compounds of the group: cisplatin, doxorubicin, gemcitabine, docetaxel, paclitaxel, bleomycin; and a corresponding pharmaceutical composition, optionally in separate containers or packages, wherein said integrin inhibitor is an antibody or a functionally intact derivative thereof, comprising a binding site which binds to an epitope of an integrin receptor, preferably selected from the group of antibodies or their bi- or monovalent derivatives (Fab′2)-(Fab′): LM609, Vitaxan, Abciximab (7E3), P1F6, 14D9.F8, CNTO95, humanized, chimeric and de-immunized versions thereof included.

In another embodiment of the present invention the chemotherapeutic agent can be melphalan or TNFα, preferably applied in combination.

It should be understood that all cancer co-therapeutic agents independent from their nature may be used in combination. It is especially preferred to use a chemotherapeutic substance, i.e. one of cisplatin, doxorubicin, gemcitabine, docetaxel, paclitaxel, bleomycin together with TNFα.

A preferred embodiment of the present invention relates to a package for use in isolated organ perfusion comprising at least one integrin ligand, preferably an αᵥβ₃, αᵥβ₅, αᵥβ₆ or αᵥβ₈ integrin receptor inhibiting agent, more preferably an RGD-containing linear or cyclic peptide, especially cyclo(Arg-Gly-Asp-DPhe-NMeVal); optionally further comprising a package comprising a cytotoxic agent.

A further preferred embodiment of the present invention relates to a corresponding pharmaceutical kit, wherein said integrin ligand is an antibody or an active derivative thereof, preferably selected from the group of antibodies: LM609, P1F6 and 14D9.F8 as well as Vitaxin, CNTO95, Abciximab.

A preferred embodiment of the present invention relates to a specific embodiment of the invention, a specific pharmaceutical kit, comprising

(i) a package comprising cyclo(Arg-Gly-Asp-DPhe-NMeVal),

(ii) a package comprising at least one chemotherapeutic agent which is selected from any of the compounds of the group: cisplatin, doxorubicin, gemcitabine, docetaxel, paclitaxel, bleomycin and 5FU, optionally in combination with TNFα.

A preferred embodiment of the present invention relates to a specific embodiment of the invention, a specific pharmaceutical kit, comprising

(i) a package comprising cyclo(Arg-Gly-Asp-DPhe-NMeVal),

(ii) a package comprising at least one chemotherapeutic agent which is selected from any of the compounds of the group: melphalan, cisplatin, doxorubicin, gemcitabine, docetaxel, paclitaxel, bleomycin and 5FU, optionally in combination with TNFα.

In a further preferred embodiment the kit comprises melphalan and/or TNFα.

A further preferred embodiment of the present invention relates the use of a pharmaceutical composition or a pharmaceutical kit as defined above, below and in the claims, for the manufacture of a medicament to treat tumors and tumor metastases via isolated organ perfusion.

A further preferred embodiment of the present invention relates to a pharmaceutical treatment or method for treating tumors or tumor metastases in a patient via isolated organ perfusion, the treatment or method comprising administering to said patient a therapeutically effective amount of an agent or agents having

(i) integrin ligand specificity, and

(ii) a cancer cotherapeutic agent

as defined above.

The cancer cotherapeutic agent optionally is a cytotoxic, preferably chemotherapeutic agent, and said agent (i) is a αᵥβ₃, αᵥβ₅ or an αᵥβ₆ integrin inhibitor or a VEGF receptor blocking agent.

A further preferred embodiment of the present invention relates to a corresponding method, wherein said integrin ligand is cyclo(Arg-Gly-Asp-DPhe-NMeVal), and is optionally administered together with a cytotoxic drug selected from the group: cisplatin, doxorubicin, gemcitabine, docetaxel, paclitaxel, bleomycin.

The pharmaceutical treatment using the pharmaceutical compositions and kits according to the invention may be accompanied, concurrently or sequentially, by a radiation therapy.

The radiation therapy may be the sole cotherapeutic agent to be applied together with the integrin ligand.

The agents can be administered concurrently or sequentially in any of said cases.

The invention relates furthermore to a new therapy form comprising the administration of an integrin ligand prior to the administration of the cancer cotherapeutic agent.

Generally, this prior application takes place 1 to 8 hours (h), preferably 1 to 5 h, and more preferably 1 to 3 h before the application of the further cancer cotherapeutic agent. Even more preferably, this prior application takes place 2 to 8 hours (h), preferably 2 to 6 h, and more preferably 2 to 4 h before the application of the further cancer cotherapeutic agent, such as 1 to 2 h, 2 to 3 h, 3 to 6 h, 2 to 5 h or 3 to 7 h before the application of the further cancer therapeutic agent. With respect to the invention, this prior application or administration is also referred to as “timed administration” or “timed application”.

As is shown by the data obtained in this respect, the effect according to the invention is achieved in non-human animals, especially rats, if this prior application preferably takes place 1 to 8 hours (h), preferably 1 to 5 h, and more preferably 1 to 3 h before the application of the further cancer cotherapeutic agent; and even more preferably this prior application takes place 2 to 8 hours (h), preferably 2 to 6 h, and more preferably 2 to 4 h before the application of the further cancer cotherapeutic agent, such as 1 to 2 h, 2 to 3 h, 3 to 6 h, 2 to 5 h or 3 to 7 h before the application of the further cancer therapeutic agent. With respect to the invention, this prior application or administration is also referred to as “timed administration” or “timed application”

However, the data from experiments with human animals preferably shows that the time of the above/below described and discussed “prior application” can be delayed or multiplied by the factor 1 to 4 and especially 2 to 4. This difference in the response or response time between non-human animals, especially rodents, such as rats, and human animals is known and extensively discussed in the art. While the applicant wishes not to be bound by this theory, he believes that this difference is at least in part caused by the different pharmacokinetic behavior of the different species, which i. a. reflects in different halflives (t_{1/2}) in the different kinds of animals. For example, for compounds such as cyclopeptides, the halflives in rats usually are in the range of 10-30 minutes, whereas the halflives in human animals for the same compounds are within 2 to 6 hours and especially 3 to 4 hours.

Accordingly, a subject of this application is a method of treatment and/or a method of manufacture has described above/below, wherein the prior application preferably takes place 1 to 32 hours (h), preferably 2 to 32 h, more preferably 2 to 24 h, even more preferably 4 to 24 h, even more preferably 6 to 20 h and especially 6 to 16 h, before the application of the further cancer cotherapeutic agent; or alternatively preferably this prior application takes place 6 to 32 hours (h), preferably 10 to 24 h, and more preferably 12 to 20 h before the application of the further cancer cotherapeutic agent. With respect to the invention, this prior application or administration is also referred to as “timed administration” or “timed application”

However, in the preferred aspect of the instant invention, the timed administration (regardless of whether the patient is a human or nonhuman animal) of the the specific integrin ligand takes place 1 to 10 hours (h), preferably 2 to 8 h, more preferably 2 to 6 h, even more preferably 3 to 8 h, even more preferably 3 to 6 h and especially 4 to 8 h prior to the application of the one or more cancer cotherapeutic agents, e.g. 1 to 2 h, 1 to 3 h, 1 to 4 h, 2 to 3 h, 2 to 4 h, 2 to 6 h, 2 to 8 h, 2 to 10 h, 3 to 4 h, 3 to 10 h, 4 to 6 h, 4 to 10 h, 5 to 8 or 5 to 10 h. This is especially preferred if the one or more cancer cotherapeutic agents comprise external beam radiation or consist of external beam radiation.

With respect to said timed administration or timed application (of the specific integrin ligand), the hours given for said prior administration or application preferably refer to the beginning or start of the respective administration or application. Accordingly, for example, an administration of the specific integrin ligand starting three hours before the application of the respective cancer cotherapeutic agent is to be regarded as a timed administration or timed application 3 h prior to the application of the one or more cancer cotherapeutic agents according to the invention, even if the specific integrin ligand is administered by i. v. Infusion that takes an hour or two hours to be completed. This definition of prior application/prior administration is in perfect concordance with the understanding of the ones skilled in the art.

Thus, it is especially preferred when the integrin ligand is administered 2 to 6 hours prior to the administration of the cotherapeutic agent. This therapy schedule applies to all the above disclosed compositions, preparations, medicaments, methods, treatments, kits, packages and kinds of cotherapeutic agents.

In especially preferred embodiments of the present invention, in order to limit the time of the isolated organ perfusion, it is possible to administer the integrin ligand, which is in general of very low systemic toxicity, systemically prior to isolated organ perfusion, preferably an a timed administration as described herein.

Therefore, one especially preferred embodiment of the present invention is the systemic administration of an integrin ligand, preferably cyclo-(Arg-Gly-Asp-DPhe-NMe-Val), and/or the pharmaceutically acceptable derivatives, solvates and salts thereof, in a timed administration as described herein, preferably 2 to 6 hours onward followed by isolated organ perfusion with said integrin ligand, preferably cyclo-(Arg-Gly-Asp-DPhe-NMe-Val), and/or the pharmaceutically acceptable derivatives, solvates and salts thereof, and a co-therapeutic agent, the administration taking place in the form as specified above including radiotherapy.

Therefore, one especially preferred embodiment of the present invention is the systemic administration of an integrin ligand, preferably cyclo-(Arg-Gly-Asp-DPhe-NMe-Val), and/or the pharmaceutically acceptable derivatives, solvates and salts thereof, in a timed administration as described herein, preferably 2 to 6 hours onward followed by isolated organ perfusion with said integrin ligand, preferably cyclo-(Arg-Gly-Asp-DPhe-NMe-Val), and/or the pharmaceutically acceptable derivatives, solvates and salts thereof, and a co-therapeutic agent, the administration taking place in the form as specified above, optionally including radiotherapy.

Therefore, one especially preferred embodiment of the present invention is the systemic administration of an integrin ligand, preferably Cilengitide, 2 to 6 hours onward followed by isolated organ perfusion with Cilengitide and a co-therapeutic agent, the administration taking place in the form as specified above including radiotherapy.

The pharmaceutical combinations and methods of the present invention provide various benefits. The combinations according to the present invention are useful in treating and preventing tumors, tumor-like and neoplasia disorders via isolated organ perfusion. Preferably, the different combined agents of the present invention are administered in combination at a low dose, that is, at a dose lower than has been conventionally used in clinical situations. A benefit of lowering the dose of the compounds, compositions, agents and therapies of the present invention administered to a mammal includes a decrease in the incidence of adverse effects associated with higher dosages. For example, by the lowering the dosage of a chemotherapeutic agent such as methotrexate, doxorubicin, gemcitabine, docetaxel, paclitaxel, bleomycin or cisplatin, a reduction in the frequency and the severity of nausea and vomiting will result when compared to that observed at higher dosages. Similar benefits are contemplated for the compounds, compositions, agents and therapies in combination with the integrin antagonists of the present invention. By lowering the incidence of adverse effects, an improvement in the quality of life of a cancer patient is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

Alternatively, the methods and combination of the present invention can also maximize the therapeutic effect at higher doses.

### DETAILED DESCRIPTION OF THE INVENTION

If not otherwise pointed out, the terms and phrases used in this invention preferably have the meanings and definitions as given below. Moreover, these definitions and meanings describe the invention in more detail, preferred embodiments included.

If not otherwise pointed out, the reference to a compound to be used according according to the invention preferably includes the reference to the pharmaceutically acceptable derivatives, solvates and salts thereof. If not otherwise pointed out, the reference to the integrin ligands, integrin antagonists, integrin agonists, as well as the reference to the cancer-cotherapeutic agents that are compounds, preferably includes the pharmaceutically acceptable derivatives, solvates and salts thereof. Even more preferably, the reference to the integrin ligand cyclo-(Arg-Gly-Asp-DPhe-NMeVal) also includes the pharmaceutically acceptable derivatives, solvates and salts thereof, more preferably the pharmaceutically solvates and salts thereof and especially preferably the pharmaceutically acceptable salts thereof, if not indicated otherwise.

By “combination therapy unit” preferably is meant a combination of at least two distinct therapy forms so combined as to form a single therapeutical concept. In a preferred embodiment of the present invention this is the combination of an integrin ligand with a further cotherapeutic agent. It is important to note that “combination therapy unit” preferably does not mean a distinct and/or single pharmaceutical composition or medicament. By way of contrast, the integrin ligand and the further cotherapeutic agent preferably may also be provided in different containers, packages, medicaments, formulations or equivalents. Equally, the combination of integrin ligand therapy with radiation therapy is preferably comprised within the meaning of “combination therapy unit”.

With “cancer-cotherapeutic agent” or “cotherapeutic agent” preferably a cytotoxic, chemotherapeutical or immunotoxic agent is meant. Equally preferred is radiotherapy.

A “receptor” or “receptor molecule” is preferably a soluble or membrane bound or membrane associated protein or glycoprotein comprising one or more domains to which a ligand binds to form a receptor-ligand complex. By binding the ligand, which may be an agonist or an antagonist the receptor is activated or inactivated and may initiate or block pathway signaling.

By “ligand” or “receptor ligand” is preferably meant a natural or synthetic compound which binds a receptor molecule to form a receptor-ligand complex. The term ligand preferably includes agonists, antagonists, and compounds with partial agonist/antagonist activity.

An “agonist” or “receptor agonist” is preferably a natural or synthetic compound which binds the receptor to form a receptor-agonist complex by activating said receptor and receptor-agonist complex, respectively, initiating a pathway signaling and further biological processes.

By “antagonist” or “receptor antagonist” is preferably meant a natural or synthetic compound, more preferably a synthetic compound, that has a biological effect opposite to that of an agonist. An antagonist binds the receptor and blocks the action of a receptor agonist by competing with the agonist for receptor. An antagonist is defined by its ability to block the actions of an agonist. A receptor antagonist may be also an antibody or an immunotherapeutically effective fragment thereof. Preferred antagonists according to the present invention are cited and discussed below.

The term “integrin antagonists/inhibitors” or “integrin receptor antagonists/inhibitors” preferably refers to a natural or synthetic molecule, more preferably a synthetic molecule, that blocks and inhibit an integrin receptor. In some cases, the term includes antagonists directed to the ligands of said integrin receptors (such as for αᵥβ₃: vitronectin, fibrin, fibrinogen, von Willebrand's factor, thrombospondin, laminin; for αᵥβ₅: vitronectin; for αᵥβ₁: fibronectin and vitronectin; for αᵥβ₆: fibronectin). Antagonists directed to the integrin receptors are preferred according to the invention. Integrin (receptor) antagonists may be natural or synthetic peptides, non-peptides, peptidomimetica, immunoglobulins, such as antibodies or functional fragments thereof, or immunoconjugates (fusion proteins). Preferred integrin inhibitors of the invention are directed to receptor of αᵥ integrins (e.g. αᵥβ₃, αᵥβ₅, αᵥβ₆ and sub-classes). Preferred integrin inhibitors are αᵥ antagonists, and in particular αᵥβ₃ antagonists. Preferred αᵥ antagonists according to the invention are RGD peptides, peptidomimetic (non-peptide) antagonists and anti-integrin receptor antibodies such as antibodies blocking αᵥ receptors.

Exemplary, non-immunological αᵥβ₃ antagonists are described in the teachings of U.S. Pat. No. 5,753,230 and U.S. Pat. No. 5,766,591. Preferred antagonists are linear and cyclic RGD-containing peptides. Cyclic peptides are, as a rule, more stable and elicit an enhanced serum half-life. The most preferred integrin antagonist of the invention is, however, cyclo-(Arg-Gly-Asp-DPhe-NMeVal) (EMD 121974, Cilengitide®, Merck KGaA, Germany; EP 0770 622) which is efficacious in blocking the integrin receptors αᵥβ₃, αᵥβ₁, αᵥβ₆, αᵥβ₈, α_{IIb}β₃, and preferably especially efficacious with respect to integrin receptors αᵥβ₃ and/or αᵥβ₅. Suitable peptidyl as well as peptidomimetic (non-peptide) antagonists of the αᵥβ₃/αᵥβ₅/αᵥβ₆ integrin receptor have been described both in the scientific and patent literature. For example, reference is made to Hoekstra and Poulter, 1998, Curr. Med. Chem. 5, 195; WO 95/32710; WO 95/37655; WO 97/01540; WO 97/37655; WO 97/45137; WO 97/41844; WO 98/08840; WO 98/18460; WO 98/18461; WO 98/25892; WO 98/31359; WO 98/30542; WO 99/15506; WO 99/15507; WO 99/31061; WO 00/06169; EP 0853 084; EP 0854 140; EP 0854 145; U.S. Pat. No. 5,780,426; and U.S. Pat. No. 6,048,861. Patents that disclose benzazepine, as well as related benzodiazepine and benzocycloheptene αᵥβ₃ integrin receptor antagonists, which are also suitable for the use in this invention, include WO 96/00574, WO 96/00730, WO 96/06087, WO 96/26190, WO 97/24119, WO 97/24122, WO 97/24124, WO 98/15278, WO 99/05107, WO 99/06049, WO 99/15170, WO 99/15178, WO 97/34865, WO 97/01540, WO 98/30542, WO 99/11626, and WO 99/15508. Other integrin receptor antagonists featuring backbone conformational ring constraints have been described in WO 98/08840; WO 99/30709; WO 99/30713; WO 99/31099; WO 00/09503; U.S. Pat. No. 5,919,792; U.S. Pat. No. 5,925,655; U.S. Pat. No. 5,981,546; and U.S. Pat. No. 6,017,926. In U.S. Pat. No. 6,048,861 and WO 00/72801 a series of nonanoic acid derivatives which are potent αᵥβ₃ integrin receptor antagonists were disclosed. Other chemical small molecule integrin antagonists (mostly vitronectin antagonists) are described in WO 00/38665. Other αᵥβ₃ receptor antagonists have been shown to be effective in inhibiting angiogenesis.

For example, synthetic receptor antagonists such as (S)-10,11-Dihydro-3-[3-(pyridin-2-ylamino)-1-propyloxy]-5H-dibenzo[a,d]cycloheptene-10-acetic acid (known as SB-265123) have been tested in a variety of mammalian model systems. (Keenan et al., 1998, Bioorg. Med. Chem. Lett. 8(22), 3171; Ward et al., 1999, Drug Metab. Dispos. 27(11), 1232). Assays for the identification of integrin antagonists suitable for use as an antagonist are described, e.g. by Smith et al., 1990, J. Biol. Chem. 265, 12267, and in the referenced patent literature. Anti-integrin receptor antibodies are also well known. Suitable anti-integrin (e.g. αᵥβ₃, αᵥβ₅, αᵥβ₆) monoclonal antibodies can be modified to encompass antigen binding fragments thereof, including F(ab)₂, Fab, and engineered Fv or single-chain antibody. One suitable and preferably used monoclonal antibody directed against integrin receptor αᵥβ₃ is identified as LM609 (Brooks et al., 1994, Cell 79, 1157; ATCC HB 9537). A potent specific anti-αᵥβ₅ antibody, P1F6, is disclosed in WO 97/45447, which is also preferred according to this invention. A further suitable αᵥβ₆ selective antibody is MAb 14D9.F8 (WO 99/37683, DSM ACC2331, Merck KGaA, Germany), which is selectively directed to the αᵥ-chain of integrin receptors. Another suitable anti-integrin antibody is the commercialized Vitaxin®.

The term “antibody” or “immunoglobulin” herein is preferably used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity. The term generally includes heteroantibodies which are composed of two or more antibodies or fragments thereof of different binding specificity which are linked together.

Depending on the amino acid sequence of their constant regions, intact antibodies can be assigned to different “antibody (immunoglobulin) classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ respectively. Preferred major class for antibodies according to the invention is IgG, in more detail IgG1 and IgG2.

Antibodies are usually glycoproteins having a molecular weight of about 150,000, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. The variable regions comprise hypervariable regions or “CDR” regions, which contain the antigen binding site and are responsible for the specificity of the antibody, and the “FR” regions, which are important with respect to the affinity/avidity of the antibody. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). The “FR” residues (frame work region) are those variable domain residues other than the hypervariable region residues as herein defined. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains. The “light chains” of antibodies from any vertebrate species can be assigned to one of two clearly distinct types, called kappa (κ) and lambda (λ), based on the amino acid sequences of their constant domains.

The term “monoclonal antibody” as used herein preferably refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. Methods for making monoclonal antibodies include the hybridoma method described by Kohler and Milstein (1975, Nature 256, 495) and in “Monoclonal Antibody Technology, The Production and Characterization of Rodent and Human Hybridomas” (1985, Burdon et al., Eds, Laboratory Techniques in Biochemistry and Molecular Biology, Volume 13, Elsevier Science Publishers, Amsterdam), or may be made by well known recombinant DNA methods (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:58, 1-597(1991), for example. The term “chimeric antibody” preferably means antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g.: U.S. Pat. No. 4,816,567; Morrison et al., Proc. Nat. Acad. Sci., USA, 81:6851-6855 (1984)). Methods for making chimeric and humanized antibodies are also known in the art. For example, methods for making chimeric antibodies include those described in patents by Boss (Celltech) and by Cabilly (Genentech) (U.S. Pat. No. 4,816,397; U.S. Pat. No. 4,816,567).

“Humanized antibodies” preferably are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397). “Antibody fragments” preferably comprise a portion of an intact antibody, preferably comprising the antigen-binding or variable region thereof. Examples of antibody fragments include Fab, Fab′, F(ab′)₂, Fv and Fc fragments, diabodies, linear antibodies, single-chain antibody molecules; and multispecific antibodies formed from antibody fragment(s). An “intact” antibody is one which comprises an antigen-binding variable region as well as a light chain constant domain (CL) and heavy chain constant domains, CH1, CH2 and CH3. Preferably, the intact antibody has one or more effector functions. Papain digestion of antibodies produces two identical antigen-binding fragments, called “Fab” fragments, each comprising a single antigen-binding site and a CL and a CH1 region, and a residual “Fc” fragment, whose name reflects its ability to crystallize readily. The “Fc” region of the antibodies comprises, as a rule, a CH2, CH3 and the hinge region of an IgG1 or IgG2 antibody major class. The hinge region is a group of about 15 amino acid residues which combine the CH1 region with the CH2-CH3 region. Pepsin treatment yields an “F(ab′)2” fragment that has two antigen-binding sites and is still capable of cross-linking antigen. “Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions (CDRs) of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. “Fab′” fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known (see e.g. Hermanson, Bioconjugate Techniques, Academic Press, 1996; U.S. Pat. No. 4,342,566). “Single-chain Fv” or “scFv” antibody fragments comprise the V, and V, domains of antibody, wherein these domains are present in a Single polypeptide chain. Preferably, the Fv polypeptide further comprises a polypeptide linker between the VH and VL domains which enables the scFv to form the desired structure for antigen binding. Single-chain FV antibodies are known, for example, from Plückthun (The Pharmacology of Monoclonal Antibodies, Vol. 113, Rosenburg and Moore eds., Springer-Verlag, New York, pp. 269-315 (1994)), WO93/16185; U.S. Pat. No. 5,571,894; U.S. Pat. No. 5,587,458; Huston et al. (1988, Proc. Natl. Acad. Sci. 85, 5879) or Skerra and Plueckthun (1988, Science 240, 1038).

“Bispecific antibodies” preferably are single, divalent antibodies (or immunotherapeutically effective fragments thereof) which have two differently specific antigen binding sites. For example the first antigen binding site is directed to an angiogenesis receptor (e.g. integrin or VEGF receptor), whereas the second antigen binding site is directed to an ErbB receptor (e.g. EGFR or Her 2). Bispecific antibodies can be produced by chemical techniques (see e.g., Kranz et al. (1981) Proc. Natl. Acad. Sci. USA 78, 5807), by “polydoma” techniques (See U.S. Pat. No. 4,474,893) or by recombinant DNA techniques, which all are known per se. Further methods are described in WO 91/00360, WO 92/05793 and WO 96/04305. Bispecific antibodies can also be prepared from single chain antibodies (see e.g., Huston et al. (1988) Proc. Natl. Acad. Sci. 85, 5879; Skerra and Plueckthun (1988) Science 240, 1038). These are analogues of antibody variable regions produced as a single polypeptide chain. To form the bispecific binding agent, the single chain antibodies may be coupled together chemically or by genetic engineering methods known in the art. It is also possible to produce bispecific antibodies according to this invention by using leucine zipper sequences. The sequences employed are derived from the leucine zipper regions of the transcription factors Fos and Jun (Landschulz et al., 1988, Science 240, 1759; for review, see Maniatis and Abel, 1989, Nature 341, 24). Leucine zippers are specific amino acid sequences about 20-40 residues long with leucine typically occurring at every seventh residue. Such zipper sequences form amphipathic α-helices, with the leucine residues lined up on the hydrophobic side for dimer formation. Peptides corresponding to the leucine zippers of the Fos and Jun proteins form heterodimers preferentially (O'Shea et al., 1989, Science 245, 646). Zipper containing bispecific antibodies and methods for making them are also disclosed in WO 92/10209 and WO 93/11162. A bispecific antibody according the invention may be an antibody, directed to VEGF receptor and αVβ3 receptor as discussed above with respect to the antibodies having single specificity.

“Heteroantibodies” preferably are two or more antibodies or antibody-binding fragments which are linked together, each of them having a different binding specificity. Heteroantibodies can be prepared by conjugating together two or more antibodies or antibody fragments. Preferred heteroantibodies are comprised of cross-linked Fab/Fab′ fragments. A variety of coupling or crosslinking agents can be used to conjugate the antibodies. Examples are protein A, carboimide, N-succinimidyl-S-acetyl-thioacetate (SATA) and N-succinimidyl-3-(2-pyridyldithio)propionate (SPDP) (see e.g., Karpovsky et al. (1984) J. EXP. Med. 160, 1686; Liu et a. (1985) Proc. Natl. Acad. Sci. USA 82, 8648). Other methods include those described by Paulus, Behring Inst. Mitt., No. 78, 118 (1985); Brennan et a. (1985) Science 30 Method:81 or Glennie et al. (1987) J. Immunol. 139, 2367. Another method uses o-phenylenedimaleimide (oPDM) for coupling three Fab′ fragments (WO 91/03493). Multispecific antibodies are in context of this invention also suitable and can be prepared, for example according to the teaching of WO 94/13804 and WO 98/50431.

The term “fusion protein” preferably refers to a natural or synthetic molecule consisting of one ore more proteins or peptides or fragments thereof having different specificity which are fused together optionally by a linker molecule. As specific embodiment the term includes fusion constructs, wherein at least one protein or peptide is a immunoglobulin or antibody, respectively or parts thereof (“immunoconjugates”).

The term “immunoconjugate” preferably refers to an antibody or immunoglobulin respectively, or a immunologically effective fragment thereof, which is fused by covalent linkage to a non-immunologically effective molecule. Preferably this fusion partner is a peptide or a protein, which may be glycosylated. Said non-antibody molecule can be linked to the C-terminal of the constant heavy chains of the antibody or to the N-terminals of the variable light and/or heavy chains. The fusion partners can be linked via a linker molecule, which is, as a rule, a 3-15 amino acid residues containing peptide. Immunoconjugates according to the invention consist of an immunoglobulin or immunotherapeutically effective fragment thereof, directed to a receptor tyrosine kinase, preferably an ErbB (ErbB1/ErbB2) receptor and an integrin antagonistic peptide, or an angiogenic receptor, preferably an integrin or VEGF receptor and TNFα or a fusion protein consisting essentially of TNFα and IFNγ or another suitable cytokine, which is linked with its N-terminal to the C-terminal of said immunoglobulin, preferably the Fc portion thereof. The term includes also corresponding fusion constructs comprising bi- or multi-specific immunoglobulins (antibodies) or fragments thereof.

The term “functionally intact derivative” preferably means according to the understanding of this invention a fragment or portion, modification, variant, homologue or a de-immunized form (a modification, wherein epitopes, which are responsible for immune responses, are removed) of a compound, peptide, protein, antibody (immunoglobulin), immunconjugate, etc., that has principally the same biological and/or therapeutic function as compared with the original compound, peptide, protein, antibody (immunoglobulin), immunconjugate, etc. However, the term includes also such derivatives, which elicit a reduced or enhanced efficacy.

The term “cytokine” is preferably a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; mouse gonadotropin-associated peptide; inhibin; activin; vascular endothelial growth factor (VEGF); integrin; thrombopoietin (TPO); nerve growth factors such as NGFβ; platelet-growth factor; transforming growth factors (TGFs) such as TGFα and TGFβ; erythropoietin (EPO); interferons such as IFNα, IFNβ, and IFNγ; colony stimulating factors such as M-CSF, GM-CSF and G-CSF; interleukins such as IL-1, IL-1a, IL-2, IL-3, IL-4, IL-5, IL-6, IL-7, IL-8, IL-9, IL-10, IL-11, IL-12; and TNFα or TNFβ. Preferred cytokines according to the invention are interferons and TNFα.

The term “cytotoxic agent” as used herein preferably refers to a substance that inhibits or prevents the function of cells and/or causes destruction of cells. The term is intended to include radioactive isotopes, chemotherapeutic agents, and toxins such as enzymatically active toxins of bacterial, fungal, plant or animal origin, or fragments thereof. The term may include also members of the cytokine family, preferably IFNγ as well as anti-neoplastic agents having also cytotoxic activity.

The term “chemotherapeutic agent” or “anti-neoplastic agent” preferably is regarded according to the understanding of this invention as a member of the class of “cytotoxic agents”, as specified above, and includes chemical agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Suitable chemotherapeutic agents according to the invention are preferably natural or synthetic chemical compounds, but biological molecules, such as proteins, polypeptides etc. are not expressively excluded. There are large numbers of anti-neoplastic agents available in commercial use, in clinical evaluation and in pre-clinical development, which could be included in the present invention for treatment of tumors/neoplasia by combination therapy with TNFα and the anti-angiogenic agents as cited above, optionally with other agents such as EGF receptor antagonists. It should be pointed out that the chemotherapeutic agents can be administered optionally together with above-said drug combination. Examples of chemotherapeutic agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

Further examples of cancer cotherapeutic agents and preferably of chemotherapeutical agents, cytotoxic agents, immunomodulating agents and/or immunotoxic agents preferably include antibodies against one or more target, preferably selected from the group consisting of HER, HER2, PDGF, PDGFR, EGF, EGFR, VEGF, VEGFR and/or VEGFR2, wherein said antibodies are preferably selected from Herceptin, Bevacizumab (rhuMAb-VEGF, Avastin®), Cetuximab (Erbitux®) and Nimotuzumab, and preferably small molecules or NCEs against one or more of said targets, preferably selected from the group consisting of Sorafenib (Nexavar®), Sunitinib (Sutent®) and ZD6474 (ZACTIMA™).

In a preferred aspect of the instant invention, the chemotherapeutical agents, cytotoxic agents, immunomodulating agents and/or immunotoxic agents are selected from one or more of the following groups:

a) alkylating agents,

b) antibiotics,

c) antimetabolites,

d) biologicals and immunomodulators,

e) hormones and antagonists thereof,

f) mustard gas derivatives,

g) alkaloids,

h) protein kinase inhibitors.

In a more preferred aspect of the instant invention, the chemotherapeutical agents, cytotoxic agents, immunomodulating agents and/or immunotoxic agents are selected from one or more of the following groups:

a) alkylating agents, selected from busulfan, melphalan, carboplatin, cisplatin, cyclophosphamide, dacarbazine, carmustine, ifosfamide and lomustine, temozolomide, altretamine,

b) antibiotics, selected from leomycin, doxorubicin, adriamycin, idarubicin, epirubicin and plicamycin,

c) antimetabolites, selected from sulfonamides, folic acid antagonists, gemcitabine, 5-fluorouracil (5-FU), leucovorine, leucovorine with 5-FU, 5-FU with calcium folinate, and leucovorin, capecitabine, mercaptopurine, cladribine, pentostatine, methotrexate, raltitrexed, pemetrexed, thioguanine, camptothecin derivatives (topotecan, irinotecan)

d) biologicals and immunomodulators, selected from interferon a2A, interleukin 2 and levamisole,

e) hormones and antagonists thereof, selected from flutamide, goserelin, mitotane and tamoxifen,

f) mustard gas derivatives, selected from melphalan, carmustine and nitrogen mustard,

g) alkaloids, selected from taxanes, docetaxel, paclitaxel, etoposide, vincristine, vinblastine and vinorelbine.

Even more preferred chemotherapeutic agents or cancer cotherapeutic agents according to the invention are selected from the group consisting of cisplatin, carboplatin, melphalan, gemcitabine, doxorubicin, docetaxel, paclitaxel (taxol) and bleomycin.

Dosings and preferably standard administration schedules for the above given cancer cotherapapeutic agents are known in the art.

Especially preferred chemotherapeutic agents or cancer cotherapeutic agents are selected from the group consisting of melphalan and TNFα.

The term “immunotoxic” preferably refers to an agent which combines the specificity of a immunomolecule .e.g. an antibody or a functional equivalent thereof with a toxic moiety, e.g. a cytotoxic function as defined above.

The terms “cancer” and “tumor” preferably refer to or describe the physiological condition in mammals that is typically characterized by unregulated cell growth. By means of the pharmaceutical compositions according of the present invention tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. More specifically the tumor is selected from the group consisting of adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma and teratoma.

In detail, the tumor is selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adeno-carcinoma, pineal cell, pituitary tumors, plasmacytoma, pseudo-sarcoma, pulmonary blastoma, renal cell carcinoma, retinoblastoma, rhabdomyo-sarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The “pharmaceutical compositions” of the invention can preferably comprise agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art. The immunotherapeutic agents according to the invention can additionally administered with adjuvants like BCG and immune system stimulators. Furthermore, the compositions may include immunotherapeutic agents or chemotherapeutic agents which contain cytotoxic effective radio labeled isotopes, or other cytotoxic agents, such as a cytotoxic peptides (e.g. cytokines) or cytotoxic drugs and the like.

The term “ pharmaceutical kit” for treating tumors or tumor metastases preferably refers to a package and, as a rule, instructions for using the reagents in methods to treat tumors and tumor metastases. A reagent in a kit of this invention is typically formulated as a therapeutic composition as described herein, and therefore can be in any of a variety of forms suitable for distribution in a kit. Such forms can include a liquid, powder, tablet, suspension and the like formulation for providing the antagonist and/or the fusion protein of the present invention. The reagents may be provided in separate containers suitable for administration separately according to the present methods, or alternatively may be provided combined in a composition in a single container in the package. The package may contain an amount sufficient for one or more dosages of reagents according to the treatment methods described herein. A kit of this invention also contains “instruction for use” of the materials contained in the package.

The term “therapeutically effective” or “therapeutically effective amount” preferably refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

As used herein. the terms “pharmaceutically acceptable” and grammatical variations thereof, as they refer to compositions, carriers, diluents and reagents, are preferably used interchangeably and preferably represent that the materials are capable of administration to or upon a mammal without the production of undesirable physiological effects such as nausea, dizziness, gastric upset and the like. The preparation of a pharmacological composition that contains active ingredients dissolved or dispersed therein is well understood in the art and need not be limited based on formulation. Typically, such compositions are prepared as injectables either as liquid solutions or suspensions, however, solid forms suitable for solution, or suspensions, in liquid prior to use can also be prepared. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents., pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as. for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Particularly preferred is the HCl salt when used in the preparation of cyclic polypeptide αv antagonists. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin. vegetable oils such as cottonseed oil, and water-oil emulsions. Typically, a therapeutically effective amount of an immunotherapeutic agent in the form of a, for example, antibody or antibody fragment or antibody conjugate is an amount such that when administered in physiologically tolerable composition is sufficient to achieve a plasma concentration of from about 0.01 microgram (μg) per milliliter (ml) to about 100 μg/ml, preferably from about 1 μg/ml to about 5 μg/ml and usually about 5 μg/ml. Stated differently the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily for one or several days. Where the immunotherapeutic agent is in the form of a fragment of a monoclonal antibody or a conjugate, the amount can readily be adjusted based on the mass of the fragment/conjugate relative to the mass of the whole antibody. A preferred plasma concentration in molarity is from about 2 micromolar (μM) to about 5 millimolar (mM) and preferably, about 100 μM to 1 mM antibody antagonist. A therapeutically effective amount of an agent according of this invention which is a non-immunotherapeutic peptide or a protein polypeptide (e.g. IFN-alpha), or other similarly-sized small molecule, is typically an amount of polypeptide such that when administered in a physiologically tolerable composition is sufficient to achieve a plasma concentration of from about 0.1 microgram (μg) per milliliter (ml) to about 200 μg/ml, preferably from about 1 μg/ml to about 150 μg/ml. Based on a polypeptide having a mass of about 500 grams per mole, the preferred plasma concentration in molarity is from about 2 micromolar (μM) to about 5 millimolar (mM) and preferably about 100 μM to 1 mM polypeptide antagonist. The typical dosage of an active agent, which is a preferably a chemical antagonist or a (chemical) chemotherapeutic agent according to the invention (neither an immunotherapeutic agent nor a non-immunotherapeutic peptide/protein) is 10 mg to 1000 mg, preferably about 20 to 200 mg, and more preferably 50 to 100 mg per kilogram body weight per day. The preferred dosage of an active agent, which is a preferably a chemical antagonist or a (chemical) chemotherapeutic agent according to the invention (neither an immunotherapeutic agent nor a non-immunotherapeutic peptide/protein) is 0.5 mg to 3000 mg per patient and day, more preferably 10 to 2500 mg per patient and per day, and especially 50 to 1000 mg per patient and per day, or, per kilogram body weight, preferably about 0.1 to 100 mg/kg, and more preferably 1 mg to 50 mg/kg, preferably per dosage unit and more preferably per day, or, per square meter of the bodysurface, preferably 0.5 mg to 2000 mg/m², more preferably 5 to 1500 mg/m², and especially 50 to 1000 mg/m², preferably per dosage unit and more preferably per day.

A preferred subject of the instant invention is the use of at least one integrin ligand, preferably at least one integrin ligand as described herein, for the manufacture of a medicament for the treatment of cancer via isolated organ perfusion. Preferably, said medicament is to be used in combination with at least one cancer-cotherapeutic agent different from the said integrin ligand. Preferably, said at least one cancer-cotherapeutic agent is selected from the chemotherapeutical agents, cytotoxic agents, immunomodulating agents and/or immunotoxic agents as described herein and more preferably from the chemotherapeutic agents as described herein. More preferably, said at least one integrin ligand comprises cyclo-(Arg-Gly-Asp-DPhe-NMe-Val) and/or a pharmaceutically acceptable derivative, solvate and/or salt thereof. Especially preferably, said at least one integrin ligand is selected from the group consisting of cyclo-(Arg-Gly-Asp-DPhe-NMe-Val), a pharmaceutically acceptable derivative thereof, a pharmaceutically solvate and a pharmaceutically acceptable salt thereof. Preferably, the isolated organ to be perfused is selected from the group consisting of liver, lung, kidney, pelvis, pleura, pancreas and limb. The cancer to be treated according to the invention is preferably selected from the cancer types or tumor types as described herein.

Thus, a preferred subject of the instant invention is the use of at least one integrin ligand, selected from the group consisting of cyclo-(Arg-Gly-Asp-DPhe-NMe-Val) and/or a pharmaceutically acceptable derivative, solvate and/or salt thereof, for the manufacture of a medicament for the treatment of cancer via isolated organ perfusion. Preferably, said medicament is to be used in combination with at least one cancer-cotherapeutic agent different from the said integrin ligand. Preferably, said at least one cancer-cotherapeutic agent is selected from the chemotherapeutical agents, cytotoxic agents, immunomodulating agents and/or immunotoxic agents as described herein, more preferably from the chemotherapeutic agents as described herein, and especially preferably from the group consisting of melphalan, cyclophosphamid, doxorubicin, cisplatin, carboplatin, gemcitabine, docetaxel, paclitaxel, bleomycin, 5FU and TNFα, the group consisting of Herceptin, Bevacizumab, Cetuximab and Nimotuzumab, and/or the group consisting of Sorafenib, Sunitinib and ZD6474 (ZACTIMA™).

Preferably, the isolated organ is selected from the group consisting of liver, lung, kidney, pelvis, pleura, pancreas and limb, preferably the liver. The cancer or tumor to be treated is preferably selected from the ones described herein and especially preferably is hepatocellular carcinoma of the liver.

The amount of cyclo-(Arg-Gly-Asp-DPhe-NMeVal), the pharmaceutically acceptable derivatives, solvates and/or salts thereof, preferably cyclo-(Arg-Gly-Asp-DPhe-NMeVal) and/or a pharmaceutically acceptable salt thereof, to be administered to a patient can be readily determined by the ones skilled in the art. However, it is preferred to administer it in the amounts given below.

Generally, the amount of cyclo-(Arg-Gly-Asp-DPhe-NMeVal) and/or a pharmaceutically acceptable salt thereof, preferably cyclo-(Arg-Gly-Asp-DPhe-NMeVal), to be administered to a patient is at least 50 mg/m², preferably at least 100 mg/m², and more preferably at least 250 mg/m², but generally below 5000 mg/m², preferably below 4000 mg/m² and especially preferably below 2500 mg/m², for example an amount of about 120 mg/m², about 240 mg/m², about 360 mg/m², about 480 mg/m², about 600 mg/m², about 1200 mg/m², about 1800 mg/m² or about 2400 mg/m², preferably at one time or at one administration. Preferably, such an amount is administered to a patient 1 to 7 times within one week, more preferably 1 to 5 times within one week and especially 1 to 3 times within one week, such as once or twice within one week.

Accordingly, the amount of cyclo-(Arg-Gly-Asp-DPhe-NMeVal) and/or a pharmaceutically acceptable salt thereof, preferably cyclo-(Arg-Gly-Asp-DPhe-NMeVal) to be administered to patient preferably lies between 300 to 8000 mg and more preferably 800 mg to 7000 mg per week.

In a preferred aspect of the invention, the amount of cyclo-(Arg-Gly-Asp-DPhe-NMeVal) and/or a pharmaceutically acceptable salt thereof, preferably cyclo-(Arg-Gly-Asp-DPhe-NMeVal), to be administered to a patient per week is administered in about equal amounts of about 500 mg (flat) or about 2000 mg (flat) for each administration. Preferably, such an amount is administered to a patient 1 to 7 times within one week, more preferably 1 to 5 times within one week and especially 1 to 3 times within one week, such as once or twice within one week.

However, depending on the kind and/or size of the isolated organ to be perfused according to the invention, it may be advantageous to apply only a part of the amounts given before per patient and per day, mg/kg of body weight and/or per square meter (m²) of the body surface of the patient, for example ½ of the above given amounts, ⅓ of the above given amounts, ¼ of the above given amounts or 1/10 of the above given amounts. This preferably refers to the cancer cotherapeutic agent. This preferably also refers to the specific integrin ligand, as long it is used exclusively or essentially exclusively in the isolated organ perfusion. The application of only a part of the amounts as described before preferably does not apply to a specific integrin ligand that is given systemically in the context of the isolated organ perfusion.

A preferred subject of the instant invention is the use of at least one integrin ligand as described herein and at least one cancer-cotherapeutic agent different from said integrin ligand as described herein for the preparation of a medicament for the treatment of cancer via isolated organ perfusion.

The preferred subject of the instant invention is the use of at least one integrin ligand and at least one cancer-cotherapeutic agent different from said integrin ligand for the treatment of cancer via isolated limb perfusion in a subject in need thereof.

An especially preferred subject of the instant invention is the use of at least one integrin ligand as described herein for the manufacture of a medicament for the treatment of cancer, preferably cancer as described herein, via isolated organ perfusion.

In the methods and/or uses described herein, the medicament is preferably to be used in combination with at least one cancer cotherapeutic agent different from said integrin ligand, preferably with at least one cancer cotherapeutic agent as described herein.

In the methods and/or uses described herein, the at least one specific integrin ligand preferably comprises or more preferably consists of cyclo-(Arg-Gly-Asp-DPhe-NMe-Val) and/or a pharmaceutically acceptable derivative, solvate and/or salt thereof.

In the methods and/or uses described herein, the isolated organ is preferably selected from the group consisting of liver, lung, kidney, pelvis, pleura, pancreas and limb.

In the methods and/or uses described herein, the at least one cancer-cotherapeutic agent different from said integrin ligand preferably comprises one or more selected from the group consisting of melphalan, cyclophosphamid, doxorubicin, cisplatin, carboplatin, gemcitabine, docetaxel, paclitaxel, bleomycin, 5FU and TNFα.

In the methods and/or uses described herein, the at least one cancer-cotherapeutic agent different from said integrin ligand preferably comprises one or more selected from the group consisting of Herceptin, Bevacizumab, Cetuximab, Nimotuzumab, Sorafenib, Sunitinib and ZD6474.

In the methods and/or uses described herein, the at least one specific integrin ligand is preferably administered in a timed administration as described herein.

### EXAMPLES

The following examples are given in order to assist the skilled artisan to better understand the present invention by way of exemplification. The examples are not intended to limit the scope of protection conferred by the claims. The features, properties and advantages exemplified for the compounds and uses defined in the examples may be assigned to other compounds and uses not specifically described and/or defined in the examples, but falling under the scope of what is defined in the claims.

### Example 1

Synergy of Cilengitide (=cyclo-(Arg-Gly-Asp-DPhe-NMeVal)) with alkylating agent melphalan in combination with or without the biological agent TNFα in therapy via isolated limb perfusion of soft tissue syngeneic rat sarcoma BN175.

Immunocompetent rats are implanted in a hind limb with the BN175 syngeneic soft tissue sarcoma. When the tumors reached a volume of 500 mm³ the limb is isolated and perfused with therapeutic substances for 20 minutes. After wash out, the limb is reconnected to the circulation, and the animal allowed to recover.

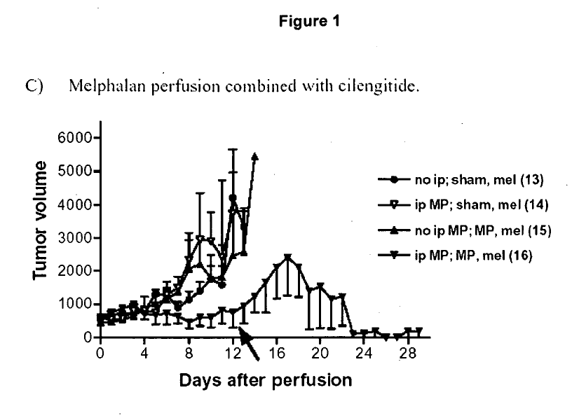

The therapy experiment involves an ip bolus and a perfusion phase. If Cilengitide (“MP”) is given as bolus (50 mg/kg) the curve is labeled “ip MP”, otherwise “no ip”. If Cilengitide is present during perfusion phase, or not is indicated by MP or Sham. All conditions contain melphalan (10 μg/ml) in perfusion, indicated by “mel”. As can be seen from the graph of FIG. 1, the combination of Cilengitide and melphalan results in a dramatic positive effect due to synergistic interaction.

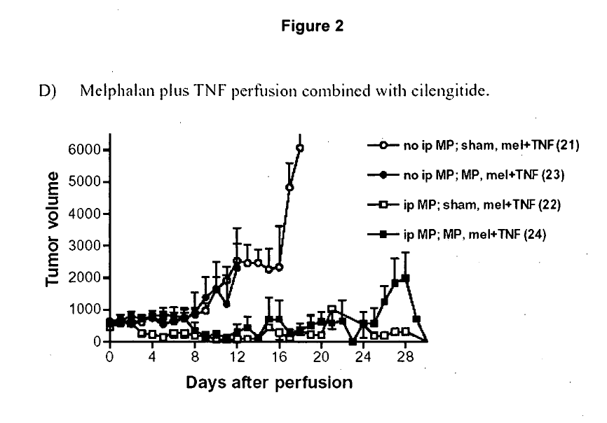

FIG. 2 coding as for FIG. 1, excepting the perfusion phase contains TNFα and melphalan (mel+TNF). As can be seen from the graphs (22) and (24) of FIG. 2 combination of Cilengitide and melphalan+TNF also results in a dramatic positive effect due to synergistic interaction (see FIG. 2 and comment below for further details).

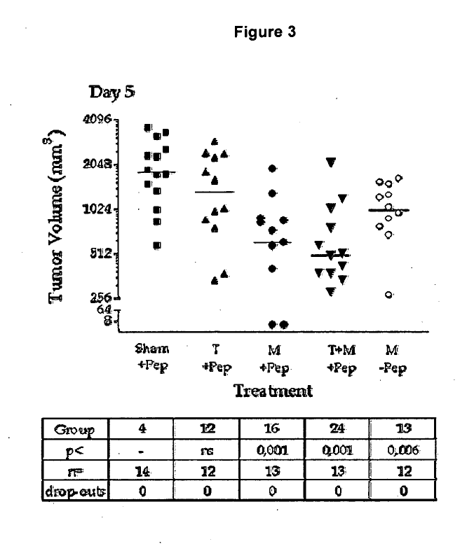

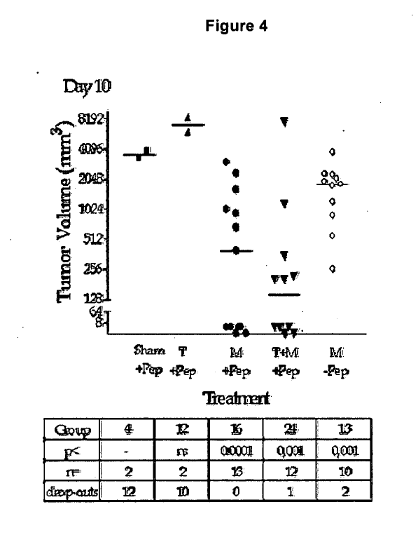

FIGS. 3 and 4 summarize the status of the individual animals included in the averaged curves of FIGS. 1 and 2 above, at days 5 and 10 after therapy, respectively.

Note the Log2 scale for tumor volume. Abbreviations are as above. In this case +/− peptide (+Pep, −Pep) refers to Cilengitide being given as bolus and in perfusion (+) or not (−), while the additions to the perfusate are given as Sham (vehicle), T (TNFα 10 μg/ml), M (melphalan−10 μg/ml), T+M (TNFα+melphalan).

By day 10 the tumors have grown so large, in the control groups, that many animals have been killed for ethical reasons.

The efficacy of the therapy in human patients corresponds to the efficacy seen at day 5 in this rat orthologous model. Efficacy at day 10 (FIG. 4) is extremely unusual, and viewed very positively by the workers.

1. A combination therapy unit for timed and combined use as a combination therapy for treatment of cancer via isolated organ perfusion, the unit comprising
a) a composition containing at least one specific integrin ligand, the unit further comprising
b) at least one further cancer-cotherapeutic agent different from the at least one specific integrin ligand of a).

2. The unit according to claim 1, wherein an isolated organ is selected from the group consisting of liver, lung, kidney, pelvis, pleura, pancreas and limb.

3. The unit according to claim 2, wherein the isolated organ is liver and the cancer to be treated is hepatocellular carcinoma.

4. The unit according to claim 1, wherein the cancer-cotherapeutic agent is radiotherapy.

5. A method for treatment of cancer comprising treating via isolated organ perfusion a subject in need thereof with a therapeutically effective amount of at least one specific integrin ligand and a further cancer-cotherapeutic agent different from said at least one specific integrin ligand.

6. The method according to claim 5, wherein the isolated organ is selected from the group consisting of liver, lung, kidney, pelvis, pleura, pancreas and limb.

7. The method according to claim 6, wherein the isolated organ is liver and cancer to be treated is hepatocellular carcinoma.

8. A set for use in isolated organ perfusion for treatment of cancer comprising independent dosage forms of
a) a therapeutically effective amount of at least one specific integrin ligand, and
b) a therapeutically effective amount of at least one further cancer-cotherapeutic agent that is not radiotherapy and is different from the at least one specific integrin ligand of a).

9. A set for use in isolated organ perfusion for treatment of cancer comprising independent dosage forms of
a) a therapeutically effective amount of at least one specific integrin ligand, and
b) a therapeutically effective amount of at least one further cancer-cotherapeutic agent, wherein the at least one further cancer-cotherapeutic agent is radiotherapy.

10. The set according to claim 8, wherein the isolated organ is liver and the cancer is hepatocellular carcinoma.

11. A method for treatment of cancer comprising administering to a subject in need thereof, via isolated organ perfusion at least one integrin ligand and at least one cancer-cotherapeutic agent different from said integrin ligand.

12. The method according to claim 11, wherein the isolated organ is a limb.

13. A method for treatment of cancer, comprising administering to a subject in need thereof, via isolated organ perfusion at least one integrin ligand.

14. The method according to claim 13, wherein the pharmaceutical composition is to be used in combination with said at least one cancer cotherapeutic agent different from said integrin ligand.

15. The method according to claim 13, wherein the at least one integrin ligand comprises cyclo-(Arg-Gly-Asp-DPhe-NMe-Val) and/or a pharmaceutically acceptable derivative, solvate and/or salt thereof.

16. A method according to claim 13, wherein the isolated organ is selected from the group consisting of liver, lung, kidney, pelvis, pleura, pancreas and limb.

17. The method according to claim 14, wherein the at least one cancer-cotherapeutic agent different from said integrin ligand comprises one or more selected from the group consisting of melphalan, cyclophosphamid, doxorubicin, cisplatin, carboplatin, gemcitabine, docetaxel, paclitaxel, bleomycin, 5FU and TNFα.

18. The method according to claim 14, wherein the at least one cancer-cotherapeutic agent different from said integrin ligand comprises one or more selected from the group consisting of Herceptin, Bevacizumab, Cetuximab, Nimotuzumab, Sorafenib, Sunitinib and ZD6474.

19. The set according to claim 9, wherein the isolated organ is liver and the cancer is hepatocellular carcinoma.
